(19) United States
(12) Patent Application Publication
(10) Pub. No.: US 2017/0135843 A1
(43) Pub. Date: May 18, 2017

(54) AN EMERGENCY SPLINT FOR SUPPORTING INJURED LIMB

(71) Applicant: Ghansham Das Agarwal, Kolkata (IN)

(72) Inventor: Ghansham Das Agarwal, Kolkata (IN)

(21) Appl. No.: 13/982,015

(22) PCT Filed: Nov. 27, 2012
(86) PCT No.: PCT/IN2012/000772

§ 371 Date: Aug. 30, 2013

(30) Foreign Application Priority Data
Dec. 7, 2011 (IN) 1236/KOL/2011

(51) Int. Cl. A61F 5/058

(52) U.S. Cl. CPC A61F 5/05841; A61F 5/05825

(57) ABSTRACT

This invention relates to an emergency splint for supporting injured limb comprising of a large part and a small part wherein the large part comprising of a plurality of longitudinal strips and the small part comprising of a plurality of transverse strips joined together such as herein described.

### FIELD OF INVENTION

This invention relates to an emergency splint for supporting injured limb.

### BACKGROUND/PRIOR ART OF INVENTION

Injured limbs require support during transportation to the medical facility. There are many places of limbs comprising of upper limbs, lower limbs, elbow, knee & ankle which require different design of support systems. Further, some require, support in angular fashion and some require support in straight fashion. This gives rise to requirement of multiple varieties of splints. However, it is not feasible to carry all varieties of splint to emergency spot.

Some emergency splints are available in market. One type is called air bag splints with many shape & size of air bags. The limb is kept in these bags and the bag is inflated to support the limb. However, the disadvantages associated with these bags are that, there are many shapes and sizes, therefore an air pump is needed to inflate the bags. Further during transit it may get punctured.

Another type of splint is called vacuum splint comprising of light plastic spheres filled in an air tight bag. The bag is provided around injured limb and thereafter, air is taken out of it. The spheres stick to each other forming a shape.

The drawbacks of aforesaid vacuum splint are as follows:
Requirement of many sizes
It is very bulky
Requirement of an external device for its operation.

Another type of splint is simple Aluminum strip lined with foal on both sides,' which is packed in roll form. While using it requires to be straightened and applied on the limb. The problem with this type of splint is that it is not strong enough to support limb during transit. Further, it offers no support in angular fashion.

Hence, there are many disadvantages with currently available splints. Many of these are not suitable for emergency situation because of requirements of multiple sizes 86 shapes and requirements of external air pumps/vacuum pump. Some of these are very bulky. Further, strip type is not rigid enough to provide proper support.

Therefore, it is required to develop a novel emergency splint, which can overcome the aforesaid drawbacks.

### OBJECTS OF THE INVENTION

A primary object of the present invention is to provide an emergency splint for supporting injured limb, which overcomes disadvantages associated with the prior arts.

Another object of the present invention is to provide an emergency splint for supporting injured limb, wherein a single size can be used in almost all situation.

Still another object of the present invention is to provide an emergency splint for supporting injured limb, which is compact and light weight.

Yet another object of the present invention is to provide an emergency splint for supporting injured limb, which can be applied in straight fashion as well as in angular fashion.

Yet another further object of the present invention is to provide an emergency splint for supporting injured limb, which can be applied to all parts of upper as well as lower limbs of body.

Yet another further object of the present invention is to provide an emergency splint for supporting injured limb, which provides enough rigid support in all fashions so as to prevent further damage to injured limb during transportation.

Still another object of the present invention is to provide an emergency splint for supporting injured limb, which can be joined with another similar splint to increase width and length.

Further object of the present invention is to provide an emergency splint for supporting injured limb, which is simple in construction and efficient.

### SUMMARY OF THE INVENTION

The instant invention provides an emergency splint comprising of following components;

1) Large part “A” is made of at least three inward curved large strips joined together with hook & loop fastener straps.
2) Small part “B” is made of at least three inward curved small strips joined together with hook & loop fastener straps.
3) A bendable part made of bendable material strip joins together part A and part B.
4) Two side flap strips with loop fastener on its distal ends are attached with side strips of part “B” with pivot & are movable in circular fashion.

### STATEMENT OF INVENTION

According to this invention, there is provided an emergency splint for supporting injured limb comprising of a large part and a small part wherein the large part comprising of a plurality of longitudinal strips and the small part comprising of a plurality of transverse strips joined together such as herein described.

### BRIEF DESCRIPTION OF THE ACCOMPANYING DRAWINGS

Further objects and advantages of this invention will be more apparent from the ensuing description when read in conjunction with the accompanying drawings and wherein:

FIG. 1 shows: emergency splint of the present invention.

FIG. 2 shows: position of various parts of splint in angular fashion.

FIG. 3 shows: position of various parts of splint in straight fashion.

FIG. 4 shows: two splints attached circumferentially.

FIG. 5 shows: two splints attached lengthwise.

### DETAILED DESCRIPTION OF THE INVENTION WITH REFERENCE TO THE ACCOMPANYING DRAWINGS

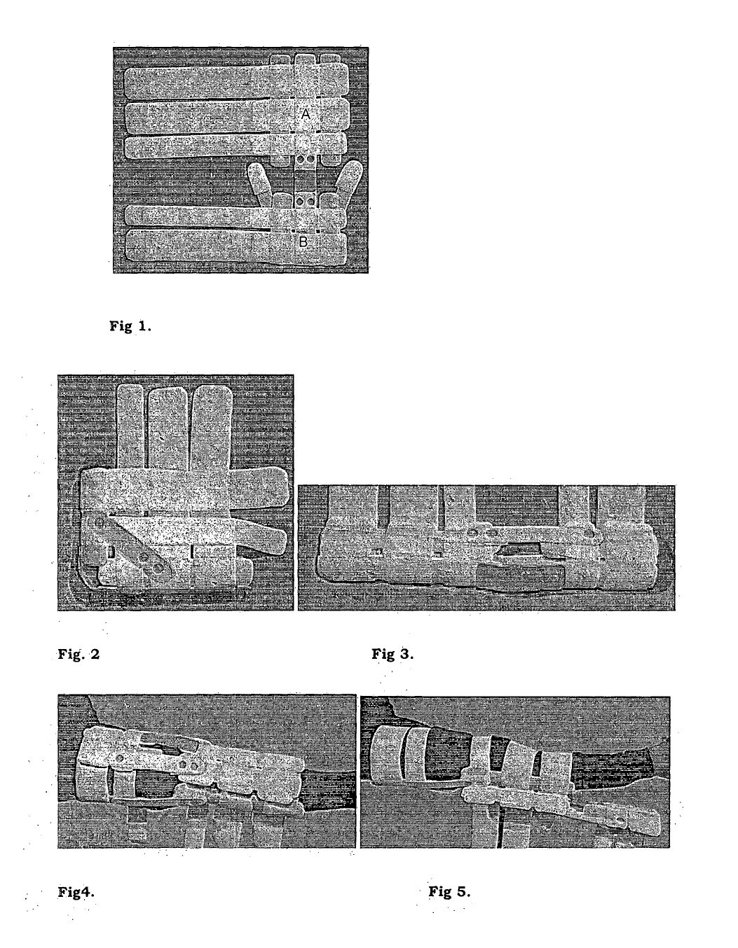

The present invention discloses an emergency splint for supporting injured limbs. Reference may be made to FIG. 1, wherein the splint comprising of two main parts i.e. large part (A) and small part (B).

The large part (A) comprises of at least three longitudinal strips joined together with loop and hook fastener straps.

Similarly, small part (B) comprises of at least three transverse strips joined together with loop and hook fastener straps.

The strips are partially curved inside, width wise. So that, these confirm to the roundness of the body limbs. The strips can be made of any rigid material such as plastic, wood, metal. The splint is provided with loop fastener inside and hook fastener outside. The loop fastener is large enough to surround the limb so as to secure the splint.

The parts A and B are joined together with a bendable strip (C). The strip (C) is such as soft flexible plastic, metal foam strip without restricting scope of the invention to the same.

The small part (B) is having at least two movable side flap strips, which are joined with strips of part B with a pivot. These side flap strips can be moved in any direction. Distal part of said side strips is provided with loop fastener attached thereto.

The splint is compact and light in weight. Therefore, it can be easily carried by army in his back pack to the front.

To use splint for bent parts of limb, the splint is bent at right angle as shown in FIG. “2”. The side flaps are attached to side strips of large part “A” giving it extra support in angular fashion.

To use splint to support straight parts, the splint is straightened and side flaps are attached with side strips of large part “B” as shown in FIG. 3.

If required two or more splints can be attached circumferentially (FIG. 4) and/or lengthwise (FIG. 5). The inner side of the splint has provision of loop fastener and outer side has hook fastener. When applied one on other, these two join because of hook 85 loop fastener. In this way, it is highly flexible to use.

It is to be noted that the present invention is susceptible to modifications, adaptations and changes by those skilled in the art. Such variant embodiments employing the concepts and features of this invention are intended to be within the scope of the present invention, which is further set forth under the following claims:

1-9. (canceled)

10. An emergency splint for supporting injured limb comprising a large part and a small part; the large part comprising a plurality of longitudinal strips and the small part comprising a plurality of transverse strips joined together by means of a bendable strip, wherein the large part comprises at least three longitudinal strips joined together with loop and hook fastener straps, and strips of said small part are joined with at least two movable side flap strips, a distal part having loop fastener attached thereto.

11. An emergency splint as claimed in claim 10, wherein the small part comprises at least three transverse strips joined together with loop and hook fastener straps.

12. An emergency splint as claimed in claim 10, wherein said strips are partially curved inside, width wise for conforming to roundness of the body limbs.

13. An emergency splint as claimed in claim 10, wherein the bendable strip comprises soft flexible plastic, metal, foam strip.

14. An emergency splint as claimed in claim 10, wherein said longitudinal/transverse strips comprise a rigid material.

15. An emergency splint as claimed in claim 14, wherein said rigid material comprises plastic, wood, or metal.

16. An emergency splint as claimed in claim 11, wherein said strips are partially curved inside, width wise for conforming to roundness of the body limbs.

17. An emergency splint as claimed in claim 11, wherein the bendable strip comprises soft flexible plastic, metal, foam strip.

18. An emergency splint as claimed in claim 11, wherein said longitudinal/transverse strips comprise a rigid material.

19. An emergency splint as claimed in claim 18, wherein said rigid material comprises plastic, wood, or metal.
